(19) United States
(12) Patent Application Publication
(10) Pub. No.: US 2009/0023712 A1
(43) Pub. Date: Jan. 22, 2009

(54) Pharmaceutical Compositions for the Treatment of Attention Deficit Hyperactivity Disorder Comprising Flibanserin

(75) Inventors: Boris Ferger, Biberach (DE); Angelo Ceci, Mittelbiberach (DE)

Correspondence Address: Michael P. Morris;Boehringer Ingelheim USA Corporation, 900 Ridgebury Road, Ridgefield, CT, 06877-0368, US

(73) Assignee: BOEHRINGER INGELHEIM INTERNATIONAL GMBH, Ingelheim (DE)

(21) Appl. No.: 12/279,589

(22) PCT Filed: Feb. 15, 2007
(86) PCT No.: PCT/EP07/51460

§ 371 Date: Sep. 26, 2008

(30) Foreign Application Priority Data
Feb. 18, 2006 (EP) 06003332.1

(51) Int. Cl. A61K 31/496; A61K 31/5513; A61P 25/00

(52) U.S. Cl. 514/220; 514/254.06; 514/254.04

(57) ABSTRACT

The invention relates to new pharmaceutical compositions for the treatment of Attention Deficit Hyperactivity Disorder (ADHD) and methods for the preparation thereof. In a preferred embodiment, the instant invention is directed to pharmaceutical combinations comprising flibanserin as one active ingredient in combination with at least one additional active ingredient for the treatment ADHD and methods for the preparation thereof.

The invention relates to new pharmaceutical compositions for the treatment and/or prevention of Attention Deficit Hyperactivity Disorder (ADHD). In a preferred embodiment, the instant invention is directed to pharmaceutical combinations comprising flibanserin, optionally in form the free base, the pharmacologically acceptable acid addition salts and/or optionally in form of the hydrates and/or solvates thereof, as one active ingredient in combination with one or more, preferably one additional active ingredient for the treatment and/or prevention of ADHD and methods for the preparation thereof.

### DESCRIPTION OF THE INVENTION

The invention relates to new pharmaceutical compositions for the treatment and/or prevention of Attention Deficit Hyperactivity Disorder (ADHD) and methods for the preparation thereof. In one embodiment, the instant invention is directed to pharmaceutical combinations comprising a therapeutically effective amount of flibanserin 1, optionally in form the free base, the pharmacologically acceptable acid addition salts and/or optionally in form of the hydrates and/or solvates thereof, as one active ingredient in combination with a therapeutically effective amount of one or more, preferably one additional active ingredient 2, optionally in form of the pharmaceutically acceptable acid addition salts, in form of the hydrates and/or solvates and optionally in the form of the individual optical isomers, mixtures of the individual enantiomers or racemates thereof, for the treatment and/or prevention of Attention Deficit Hyperactivity Disorder (ADHD) and methods for the preparation thereof.

Attention Deficit Hyperactivity Disorder (ADHD) is a disorder which may be divided into four subtypes, according to the main features associated with the disorder: inattentiveness, impulsivity, and hyperactivity. The four subtypes are ADHD predominantly impulsivity type, ADHD predominantly inattentive type, ADHD predominantly hyperactive-impulsive type and ADHD not otherwise specified (e.g. Attention Deficit Disorder (ADD)).

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

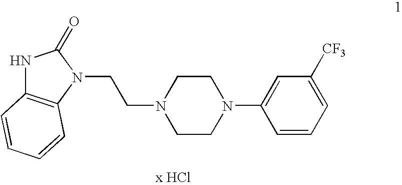

Flibanserin shows affinity for the 5-HT_{1A} and 5-HT₂-receptor. It is therefore a promising therapeutic agent for the treatment of a variety of diseases, for instance depression, schizophrenia, Parkinson, anxiety, sleep disturbances, sexual and mental disorders and age associated memory impairment.

A further embodiment of the invention is directed to pharmaceutical compositions comprising a therapeutically effective amount of flibanserin 1 in combination with a therapeutically effective amount of one or more, preferably one active ingredient 2 selected from the group consisting of adrenoceptor agonists, noradrenaline reuptake inhibitors, dopamine agonists and dopamine antagonists.

A further embodiment of the invention is directed to pharmaceutical compositions comprising a therapeutically effective amount of flibanserin 1 in combination with a therapeutically effective amount of one or more, preferably one active ingredient 2 selected from the group consisting of adrenoceptor agonists, noradrenaline reuptake inhibitors and dopamine antagonists.

A further embodiment of the invention is directed to pharmaceutical compositions comprising a therapeutically effective amount of flibanserin 1 in combination with a therapeutically effective amount of one or more, preferably one active ingredient 2 selected from the group consisting of adrenoceptor agonists and noradrenaline reuptake inhibitors.

A further embodiment of the invention is directed to pharmaceutical compositions comprising a therapeutically effective amount of flibanserin 1 in combination with a therapeutically effective amount of one or more, preferably one active ingredient 2 selected from the group consisting of noradrenaline reuptake inhibitors.

The compositions according to the invention may contain flibanserin 1 and the one or more additional active ingredient 2 in a single formulation or in separate formulations. If flibanserin and the one or more additional active ingredient are present in separate formulations these separate formulations may be administered simultaneously or sequentially.

A further embodiment according to the invention is directed to pharmaceutical compositions comprising a therapeutically effective amount of flibanserin 1 and a therapeutically effective amount of one or more, preferably one adrenoceptor agonist 2a, optionally in combination with a pharmaceutically acceptable excipient. Examples of suitable adrenoceptor agonists include Lisdexamphetamine dimesylate, Amphetamine, Dexamphetamine, NRP-104, SPD-465 (guanfacine) and Modafinil, optionally in form of the pharmaceutically acceptable acid addition salts, in form of the hydrates and/or solvates and optionally in the form of the individual optical isomers, mixtures of the individual enantiomers or racemates thereof.

A further embodiment according to the invention is directed to pharmaceutical compositions comprising a therapeutically effective amount of flibanserin 1 and a therapeutically effective amount of one or more, preferably one noradrenaline reuptake inhibitor 2b, optionally in combination with a pharmaceutically acceptable excipient. Examples of suitable noradrenaline reuptake inhibitor, include Atomoxetine, DOV-102677, DOV-216303, DOV-21947, NS 2359, NS 2330, NS 2360 and NS2390, optionally in form of the pharmaceutically acceptable salts, in form of the hydrates and/or solvates and optionally in the form of the individual optical isomers, mixtures of the individual enantiomers or racemates thereof.

A further embodiment according to the invention is directed to pharmaceutical compositions comprising a therapeutically effective amount of flibanserin 1 and a therapeutically effective amount of one or more, preferably one dopamine agonists 2c, optionally in combination with a pharmaceutically acceptable excipient. Examples of suitable dopamine agonists include Methylphenidate, Dexmethylphenidate, SPD-485, SPD-465, SPD-483, Hydroxybupropion, Pramipexol, ABT-724, CP-226269, Dextroampthemine Bromocriptin, Cabergolin, Alpha-dihydroergocryptin, Lisuride, Pergolide, Roxindol, Ropinirol, Sopirinol, Talipexol, Bupropion, Bifemelane (SON-216) and Terguride, optionally in form of the pharmaceutically acceptable acid addition salts, in form of the hydrates and/or solvates and optionally in the form of the individual optical isomers, mixtures of the individual enantiomers or racemates thereof.

Preferred dopamine agonists 2c are selected from the group consisting of Methylphenidate, Dexmethylphenidate, Hydroxybupropion, Pramipexol, Pergolide, Ropinirol, Talipexol, Bifemelane (SON-216) and Bupropion, optionally in form of the pharmaceutically acceptable acid addition salts, in form of the hydrates and/or solvates and optionally in the form of the individual optical isomers, mixtures of the individual enantiomers or racemates thereof.

More preferred dopamine agonists 2c are selected from the group consisting of Methylphenidate, Dexmethylphenidate and Hydroxybupropion, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one dopamine antagonist 2d, optionally in combination with a pharmaceutically acceptable excipient. Examples of suitable dopamine antagonist 2d include Olanzapine, Ziprasidone, MDL-814608A, NRA-0562, S-18126, SPI-376, YM-50001, 1192U90, ALX-D4, Balaperidone, CI-1030, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, Mianserin, Buspirone, PD-089232, PD-108306, PD-165167, PD-167063, PD-168306, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E and YM-43611, optionally in form of the pharmaceutically acceptable acid addition salts, in form of the hydrates and/or solvates and optionally in the form of the individual optical isomers, mixtures of the individual enantiomers or racemates thereof.

Preferred dopamine antagonist 2d are selected from the group consisting of Olanzapine and Ziprasidone, optionally in form of the pharmaceutically acceptable acid addition salts, in form of the hydrates and/or solvates and optionally in the form of the individual optical isomers, mixtures of the individual enantiomers or racemates thereof.

Flibanserin 1 may be used in form of the free base, optionally in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin 1 is used in form of the free base, it is preferably used in form of flibanserin polymorph A as disclosed in WO 03/014079.

The active ingredients 2 which are suitable to be combined with flibanserin within the teaching of the instant invention and which are mentioned hereinbefore may also be capable of forming acid addition salts with pharmaceutically acceptable acids. Representative salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds 2 carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds 2 may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

The present invention includes within its scope prodrugs of the compounds 1 and 2. In general, such prodrugs will be functional derivatives of the compounds of this invention which are readily convertible in vivo into the required compound.

The term “therapeutically effective amount” shall mean that amount of a drug or pharmaceutical agent that will elicit the biological or medical response of a tissue, system, animal or human that is being sought by a researcher or clinician. As used herein, the term “composition” is intended to encompass a product comprising the specified ingredients in the specified amounts, as well as any product which results, directly or indirectly, from combination of the specified ingredients in the specified amounts.

In the combination of the present invention, the components 1 and 2 may be administered separately or together in one pharmaceutical composition. In addition, the administration of one element of the combination of the present invention may be prior to, concurrent to, or subsequent to the administration of the other element of the combination.

The elements of the combination of 1 and 2 may be administered by oral, parenteral (e.g., intramuscular, intraperitoneal, intravenous or subcutaneous injection, or implant), buccal, nasal, rectal, sublingual or topical routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the components 1 and 2 of this invention may conveniently be presented in dosage unit form and may be prepared by any of the methods well known in the art of pharmacy. Al methods include the step of bringing the active ingredient into association with the carrier which is constituted of one or more accessory ingredients. In general, the pharmaceutical compositions are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical compositions containing the active ingredients 1 and 2, separately or together, that are suitable for oral administration may be in the form of discrete units such as hard or soft capsules, tablets, troches or lozenges, each containing a predetermined amount of the active ingredients; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Dosage forms intended for oral use may be prepared according to any method known to the art for the manufacture of pharmaceutical formulations and such compositions.

The excipients used may be for example, (a) inert diluents such as mannitol, sorbitol, calcium carbonate, pregelatinized starch, lactose, calcium phosphate or sodium phosphate; (b) granulating and disintegrating agents, such as povidone, copovidone, hydroxypropylmethylcellulose, corn starch, alginic acid, crospovidone, sodiumstarchglycolate, croscarmellose, or polacrilin potassium; (c) binding agents such as microcrystalline cellulose or acacia; and (d) lubricating agents such as magnesium stearate, stearic acid, fumaric acid or talc.

In some cases, formulations for oral use may be in the form of hardgelatin or HPMC capsules wherein the active ingredient 1 or 2, separately or together, is mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, perfuming and preserving agents.

Aqueous suspensions normally contain the active materials 1 and 2, separately or together, in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredients 1 and 2, separately or together, in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredients 1 and 2, separately or together, in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegetable oil such as olive oil or arachis oils, or a mineral oil such as liquid paraffin or a mixture thereof.

Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

The pharmaceutical compositions containing 1 and 2, separately or together, may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Preparations according to this invention containing 1 and 2, separately or together, for parenteral administration include sterile aqueous or non-aqueous solutions, suspension, or emulsions.

Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be reconstituted in sterile water, or some other sterile injectable medium immediately before use. The combination of this invention may also be administered in the form of suppositories for rectal administration. This composition can be prepared by mixing the drugs with a suitable non-irritating excipient which is solid at ordinary temperatures but liquid at the rectal temperature and will therefore melt in the rectum to release the drug. Such materials are cocoa butter, hard fat, and polyethylene glycols. Compositions for buccal, nasal or sublingual administration are also prepared with standard excipients well known in the art.

For topical administration the combinations of this invention containing 1 and 2, separately or together, may be formulated in liquid or semi-liquid preparations such as liniments, lotions, applications; oil-in-water or water-in-oil emulsions such as creams, ointments, jellies or pastes, including tooth-pastes; or solutions or suspensions such as drops, and the like.

The dosage of the active ingredients in the compositions of this invention may be varied. However, it is necessary that the amount of the active ingredients 1 and 2 be such that a suitable dosage form is obtained. The selected dosage and the dosage form depend upon the desired therapeutic effect, on the route of administration and on the duration of the treatment. Dosage ranges in the combination are approximately one tenth to one times the clinically effective ranges required to induce the desired therapeutic effect, respectively when the compounds are used singly.

Within the instant invention flibanserin 1 is preferably administered in such an amount that per single dosage between 1 to 200 mg of flibanserin 1 are applied. Preferred are ranges of between 2 to 150 mg, particular preferred 5 to 100 mg of flibanserin 1. Suitable dosage forms may contain for instance 1, 5, 10, 15, 20, 25, 30, 35, 40, 45, 50, 55, 60, 65, 70, 75, 80, 85, 90, 95 or 100 mg of flibanserin 1. The aforementioned values are based on flibanserin 1 in form of the free base. If flibanserin 1 is applied in form of one of its acid addition salts, the corresponding values are readily calculable from the aforementioned values.

Within the instant invention the adrenoceptor agonist 2a is preferably administered in such an amount that per day between 0.01 to 400 mg of 2a are applied. Preferred are ranges of between 0.05 to 300 mg, particular preferred 0.25 to 250 mg of 2a. In case of the preferred adrenoceptor agonist 2a Lisdexamphetamine dimesylate particularly preferred doses per day are in the range of about 0.5 to 100 mg. In case of the preferred adrenoceptor agonist 2a Amphetamine particularly preferred doses per day are in the range of about 0.25 to 75 mg. In case of the preferred adrenoceptor agonist 2a Dexamphetamine particularly preferred doses per day are in the range of about 0.1 to 40 mg. In case of the preferred adrenoceptor agonist 2a NRP-104 particularly preferred doses per day are in the range of about 0.1 to 100 mg. In case of the preferred adrenoceptor agonist 2a Modafinil particularly preferred doses per day are in the range of about 0.5 to 150 mg. Suitable dosage forms may contain for instance 0.01, 0.02, 0.03, 0.04, 0.05, 0.075, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395 or 400 mg of 2a. Advantageously, the compounds 2a of the present invention may be administered in a single daily dose, or the total daily dosage may be administered in divided doses of two, three or four times daily.

Within the instant invention the noradrenaline reuptake inhibitor 2b is preferably administered in such an amount that per day between 0.01 to 500 mg of 2b are applied. Preferred are ranges of between 0.025 to 450 mg, particular preferred 0.1 to 400 mg of 2b. In case of the preferred noradrenaline reuptake blocker 2b Atomoxetine particularly preferred doses per day are in the range of about 1 to 200 mg. In case of the preferred noradrenaline reuptake blocker 2b DOV-102677, DOV-216303, and NS2390 particularly preferred doses per day are in the range of about 1 to 400 mg. Suitable dosage forms may contain for instance 0.01, 0.02, 0.03, 0.04, 0.05, 0.075, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395 or 400 mg of 2b. Advantageously, the compounds 2b of the present invention may be administered in a single daily dose, or the total daily dosage may be administered in divided doses of two, three or four times daily.

Within the instant invention the dopamine agonists 2c is preferably administered in such an amount that per day between 0.01 to 1000 mg of 2c are applied. Preferred are ranges of between 0.025 to 600 mg, particular preferred 0.1 to 500 mg of 2c. In case of the preferred dopamine agonists 2c Methylphenidate particularly preferred doses per day are in the range of about 0.1 to 500 mg. In case of the preferred dopamine agonist 2c Dexmethylphenidate particularly preferred doses per day are in the range of about 1 to 300 mg. In case of the preferred dopamine agonist 2c Pramipexole particularly preferred doses per day are in the range of about 0.1 to 10 mg. In case of the preferred dopamine agonist 2c Ropinirole particularly preferred doses per day are in the range of about 0.05 to 30 mg. In case of the preferred dopamine agonist 2c Hydroxybupropion particularly preferred doses per day are in the range of about 1 to 50 mg. In case of the preferred dopamine agonist 2c Bupropion particularly preferred doses per day are in the range of about 100 to 450 mg. In case of the preferred dopamine agonist 2c Pergolide particularly preferred doses per day are in the range of about 0.05 to 3 mg. In case of the preferred dopamine agonist 2c Talipexol particularly preferred doses per day are in the range of about 0.05 to 30 mg.

Suitable dosage forms may contain for instance 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 500, 550, 600, 650, 700, 750, 800, 850, 900, 950 or 1000 mg of 2c. Advantageously, the compounds 2c of the present invention may be administered in a single daily dose, or the total daily dosage may be administered in divided doses of two, three or four times daily.

Within the instant invention the dopamine antagonist 2d is preferably administered in such an amount that per day between 0.1 to 300 mg of 2d are applied. Preferred are ranges of between 1 to 250 mg, particular preferred 5 to 200 mg of 2d. In case of the preferred dopamine antagonist 2d Olanzapine particularly preferred doses per day are in the range of about 5 to 15 mg. In case of the preferred antagonist 2d Ziprasidone particularly preferred doses per day are in the range of about 20 to 80 mg. Suitable dosage forms may contain for instance 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 mg of 2d. Advantageously, the compounds 2d of the present invention may be administered in a single daily dose, or the total daily dosage may be administered in divided doses of two, three or four times daily.

In another embodiment the invention relates to a method for the treatment and/or prevention of Attention Deficit Hyperactivity Disorder (ADHD), comprising the administration of a therapeutically effective amount of flibanserin 1, optionally in form the free base, the pharmacologically acceptable acid addition salts and/or optionally in form of the hydrates and/or solvates thereof, in combination with a therapeutically effective amount of one or more, preferably one, active ingredient 2 suitable for the treatment and/or prevention of ADHD, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another embodiment the invention relates to a method for the treatment and/or prevention of Attention Deficit Hyperactivity Disorder (ADHD) of predominantly impulsivity type, comprising the administration of a therapeutically effective amount of flibanserin 1, optionally in form the free base, the pharmacologically acceptable acid addition salts and/or optionally in form of the hydrates and/or solvates thereof, in combination with a therapeutically effective amount of one or more, preferably one, active ingredient 2 suitable for the treatment and/or prevention of ADHD, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another embodiment the invention relates to a method for the treatment and/or prevention of Attention Deficit Hyperactivity Disorder (ADHD) of predominantly inattentive type, comprising the administration of a therapeutically effective amount of flibanserin 1, optionally in form the free base, the pharmacologically acceptable acid addition salts and/or optionally in form of the hydrates and/or solvates thereof, in combination with a therapeutically effective amount of one or more, preferably one, active ingredient 2 suitable for the treatment and/or prevention of ADHD, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another embodiment the invention relates to a method for the treatment and/or prevention of Attention Deficit Hyperactivity Disorder (ADHD) of predominantly hyperactive-impulsive type, comprising the administration of a therapeutically effective amount of flibanserin 1, optionally in form the free base, the pharmacologically acceptable acid addition salts and/or optionally in form of the hydrates and/or solvates thereof, in combination with a therapeutically effective amount of one or more, preferably one, active ingredient 2 suitable for the treatment and/or prevention of ADHD, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another embodiment the invention relates to a method for the treatment and/or prevention of Attention Deficit Hyperactivity Disorder (ADHD) not otherwise specified (e.g. Attention Deficit Disorder (ADD)), comprising the administration of a therapeutically effective amount of flibanserin 1, optionally in form the free base, the pharmacologically acceptable acid addition salts and/or optionally in form of the hydrates and/or solvates thereof, in combination with a therapeutically effective amount of one or more, preferably one, active ingredient 2 suitable for the treatment and/or prevention of ADHD, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

Another embodiment of the invention relates to the use of the combinations of flibanserin 1, optionally in form the free base, the pharmacologically acceptable acid addition salts and/or optionally in form of the hydrates and/or solvates thereof and one or more, preferably one active ingredient 2, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment and/or prevention of any of the aforementioned disorders.

Another embodiment of the present invention relates to the use of flibanserin 1, optionally in form of the free base or in form of the pharmacologically acceptable acid addition salts thereof, in the manufacture of a medicament for the treatment of any of the aforementioned disorders in combination with one or more, preferably one active ingredient 2, optionally in form of the pharmaceutically acceptable salts, in form of the hydrates and/or solvates and optionally in the form of the individual optical isomers, mixtures of the individual enantiomers or racemates thereof.

Another embodiment of the invention is directed to the aforementioned methods and uses wherein 2 is selected from the group consisting of adrenoceptor agonists 2a, noradrenaline reuptake inhibitors 2b, dopamine agonists 2c and dopamine antagonists 2d.

Another embodiment of the invention is directed to the method for the prevention and/or treatment of any of the aforementioned diseases selected form the group consisting of Attention Deficit Hyperactivity Disorder (ADHD), ADHD of predominantly impulsivity type, ADHD of predominantly inattentive type, ADHD of predominantly hyperactive-impulsive type and ADHD not otherwise specified, comprising the administration of a therapeutically effective amount of Flibanserin 1, optionally in form of the free base or in form of the pharmacologically acceptable acid addition salts thereof, in combination with a therapeutically effective amount of one or more, preferably one adrenoceptor agonist 2a, selected from the group consisting of Lisdexamphetamine dimesylate, Amphetamine, Dexamphetamine, NRP-104, SPD-465 (guanfacine) and Modafinil, optionally in form of the pharmaceutically acceptable acid addition salts, in form of the hydrates and/or solvates and optionally in the form of the individual optical isomers, mixtures of the individual enantiomers or racemates thereof, separately or together in within one pharmaceutical composition.

Another embodiment of the invention relates to the use of the combinations of flibanserin 1, optionally in form the free base, the pharmacologically acceptable acid addition salts and/or optionally in form of the hydrates and/or solvates thereof and one or more, preferably one adrenoceptor agonist 2a, selected from the group consisting of Lisdexamphetamine dimesylate, Amphetamine, Dexamphetamine, NRP-104, SPD-465 (guanfacine) and Modafinil, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment and/or prevention of any of the aforementioned disorders. Another embodiment of the present invention relates to the use of flibanserin 1, optionally in form of the free base or in form of the pharmacologically acceptable acid addition salts thereof, in the manufacture of a medicament for the treatment of any of the aforementioned disorders in combination with one or more, preferably one adrenoceptor agonist 2a, selected from the group consisting of Lisdexamphetamine dimesylate, Amphetamine, Dexamphetamine, NRP-104, SPD-465 (guanfacine) and Modafinil, optionally in form of the pharmaceutically acceptable salts, in form of the hydrates and/or solvates and optionally in the form of the individual optical isomers, mixtures of the individual enantiomers or racemates thereof.

Another embodiment of the invention is directed to the method for the prevention and/or treatment of any of the aforementioned diseases selected form the group consisting of Attention Deficit Hyperactivity Disorder (ADHD), ADHD of predominantly impulsivity type, ADHD of predominantly inattentive type, ADHD of predominantly hyperactive-impulsive type and ADHD not otherwise specified, comprising the administration of a therapeutically effective amount of Flibanserin 1, optionally in form of the free base or in form of the pharmacologically acceptable acid addition salts thereof, in combination with a therapeutically effective amount of one or more, preferably one noradrenaline reuptake inhibitor 2b, selected from the group consisting of Atomoxetine, DOV-102677, DOV-216303, DOV-21947, NS 2359, NS 2330, NS 2360 and NS2390, optionally in form of the pharmaceutically acceptable acid addition salts, in form of the hydrates and/or solvates and optionally in the form of the individual optical isomers, mixtures of the individual enantiomers or racemates thereof, separately or together in within one pharmaceutical composition.

Another embodiment of the invention relates to the use of the combinations of flibanserin 1, optionally in form the free base, the pharmacologically acceptable acid addition salts and/or optionally in form of the hydrates and/or solvates thereof and one or more, preferably one noradrenaline reuptake inhibitor 2b, selected from the group consisting of Atomoxetine, DOV-102677, DOV-216303, DOV-21947, NS 2359, NS 2330, NS 2360 and NS2390, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment and/or prevention of any of the aforementioned disorders.

Another embodiment of the present invention relates to the use of flibanserin 1, optionally in form of the free base or in form of the pharmacologically acceptable acid addition salts thereof, in the manufacture of a medicament for the treatment of any of the aforementioned disorders in combination with one or more, preferably one noradrenaline reuptake inhibitor 2b, selected from the group consisting of Atomoxetine, DOV-102677, DOV-216303, DOV-21947, NS 2359, NS 2330, NS 2360 and NS2390, optionally in form of the pharmaceutically acceptable salts, in form of the hydrates and/or solvates and optionally in the form of the individual optical isomers, mixtures of the individual enantiomers or racemates thereof.

Another embodiment of the invention is directed to the method for the prevention and/or treatment of any of the aforementioned diseases selected form the group consisting of Attention Deficit Hyperactivity Disorder (ADHD), ADHD of predominantly impulsivity type, ADHD of predominantly inattentive type, ADHD of predominantly hyperactive-impulsive type and ADHD not otherwise specified, comprising the administration of a therapeutically effective amount of Flibanserin 1, optionally in form of the free base or in form of the pharmacologically acceptable acid addition salts thereof, in combination with a therapeutically effective amount of one or more, preferably one dopamine agonists 2c, selected from the group consisting of Methylphenidate, Dexmethylphenidate, SPD-485, SPD-465, SPD-483, Hydroxybupropion, Pramipexol, ABT-724, CP-226269, Dextroampthemine Bromocriptin, Cabergolin, Alpha-dihydroergocryptin, Lisuride, Pergolide, Roxindol, Ropinirol, Sopirinol, Talipexol, Bupropion, Bifemelane (SON-216) and Terguride, optionally in form of the pharmaceutically acceptable acid addition salts, in form of the hydrates and/or solvates and optionally in the form of the individual optical isomers, mixtures of the individual enantiomers or racemates thereof, separately or together in within one pharmaceutical composition. Preferably, the dopamine agonists 2c is selected from the group consisting of Methylphenidate, Dexmethylphenidate, Hydroxybupropion, Pramipexol, Pergolide, Ropinirol, Talipexol, Bifemelane (SON-216) and Bupropion, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of the combinations of flibanserin 1, optionally in form the free base, the pharmacologically acceptable acid addition salts and/or optionally in form of the hydrates and/or solvates thereof and one or more, preferably one dopamine agonist 2c, selected from the group consisting of Methylphenidate, Dexmethylphenidate, SPD-485, SPD-465, SPD-483, Hydroxybupropion, Pramipexol, ABT-724, CP-226269, Dextroampthemine Bromocriptin, Cabergolin, Alpha-dihydroergocryptin, Lisuride, Pergolide, Roxindol, Ropinirol, Sopirinol, Talipexol, Bupropion, Bifemelane (SON-216) and Terguride, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment and/or prevention of any of the aforementioned disorders. Preferably, the dopamine agonists 2c is selected from the group consisting of Methylphenidate, Dexmethylphenidate, Hydroxybupropion, Pramipexol, Pergolide, Ropinirol, Talipexol, Bifemelane (SON-216) and Bupropion, optionally in form of the pharmaceutically acceptable acid addition salts, in form of the hydrates and/or solvates and optionally in the form of the individual optical isomers, mixtures of the individual enantiomers or racemates thereof.

Another embodiment of the present invention relates to the use of flibanserin 1, optionally in form of the free base or in form of the pharmacologically acceptable acid addition salts thereof, in the manufacture of a medicament for the treatment of any of the aforementioned disorders in combination with one or more, preferably one dopamine agonist 2c, selected from the group consisting of Methylphenidate, Dexmethylphenidate, SPD-485, SPD-465, SPD-483, Hydroxybupropion, Pramipexol, ABT-724, CP-226269, Dextroampthemine Bromocriptin, Cabergolin, Alpha-dihydroergocryptin, Lisuride, Pergolide, Roxindol, Ropinirol, Sopirinol, Talipexol, Bupropion, Bifemelane (SON-216) and Terguride, optionally in form of the pharmaceutically acceptable salts, in form of the hydrates and/or solvates and optionally in the form of the individual optical isomers, mixtures of the individual enantiomers or racemates thereof. Preferably, the dopamine agonists 2c is selected from the group consisting of Methylphenidate, Dexmethylphenidate, Hydroxybupropion, Pramipexol, Pergolide, Ropinirol, Talipexol, Bifemelane (SON-216) and Bupropion, optionally in form of the pharmaceutically acceptable acid addition salts, in form of the hydrates and/or solvates and optionally in the form of the individual optical isomers, mixtures of the individual enantiomers or racemates thereof.

Another embodiment of the invention is directed to the method for the prevention and/or treatment of any of the aforementioned diseases selected form the group consisting of Attention Deficit Hyperactivity Disorder (ADHD), ADHD of predominantly impulsivity type, ADHD of predominantly inattentive type, ADHD of predominantly hyperactive-impulsive type and ADHD not otherwise specified, comprising the administration of a therapeutically effective amount of Flibanserin 1, optionally in form of the free base or in form of the pharmacologically acceptable acid addition salts thereof, in combination with a therapeutically effective amount of one or more, preferably one dopamine antagonist 2d, selected from the group consisting of Olanzapine, Ziprasidone, MDL-814608A, NRA-0562, S-18126, SPI-376, YM-50001, 1192U90, ALX-D4, Balaperidone, CI-1030, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, Mianserin, Buspirone, PD-089232, PD-108306, PD-165167, PD-167063, PD-168306, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E and YM-43611, optionally in form of the pharmaceutically acceptable acid addition salts, in form of the hydrates and/or solvates and optionally in the form of the individual optical isomers, mixtures of the individual enantiomers or racemates thereof, separately or together in within one pharmaceutical composition. Preferably the dopamine antagonist 2d is selected from the group consisting of Olanzapine and Ziprasidone, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of the combinations of flibanserin 1, optionally in form the free base, the pharmacologically acceptable acid addition salts and/or optionally in form of the hydrates and/or solvates thereof and one or more, preferably one dopamine antagonist 2d, selected from the group consisting of Olanzapine, Ziprasidone, MDL-814608A, NRA-0562, S-18126, SPI-376, YM-50001, 1192U90, ALX-D4, Balaperidone, CI-1030, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, Mianserin, Buspirone, PD-089232, PD-108306, PD-165167, PD-167063, PD-168306, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E and YM-43611, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment and/or prevention of any of the aforementioned disorders. Preferably the dopamine antagonist 2d is selected from the group consisting of Olanzapine and Ziprasidone, optionally in form of the pharmaceutically acceptable acid addition salts, in form of the hydrates and/or solvates and optionally in the form of the individual optical isomers, mixtures of the individual enantiomers or racemates thereof.

Another embodiment of the present invention relates to the use of flibanserin 1, optionally in form of the free base or in form of the pharmacologically acceptable acid addition salts thereof, in the manufacture of a medicament for the treatment of any of the aforementioned disorders in combination with one or more, preferably one dopamine antagonist 2d, selected from the group consisting of Olanzapine, Ziprasidone, MDL-814608A, NRA-0562, S-18126, SPI-376, YM-50001, 1192U90, ALX-D4, Balaperidone, CI-1030, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, Mianserin, Buspirone, PD-089232, PD-108306, PD-165167, PD-167063, PD-168306, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E and YM-43611, optionally in form of the pharmaceutically acceptable salts, in form of the hydrates and/or solvates and optionally in the form of the individual optical isomers, mixtures of the individual enantiomers or racemates thereof. Preferably the dopamine antagonist 2d is selected from the group consisting of Olanzapine and Ziprasidone, optionally in form of the pharmaceutically acceptable acid addition salts, in form of the hydrates and/or solvates and optionally in the form of the individual optical isomers, mixtures of the individual enantiomers or racemates thereof.

The methods of the present invention are effective in the treatment of children, adolescents or adults. For purposes of the present invention, a child is considered to be a patient below the age of puberty, an adolescent is considered to be a patient from the age of puberty up to about 18 years of age, and an adult is considered to be a patient of 18 years or older.

The following examples demonstrate possible pharmaceutical compositions comprising flibanserin in combination with one of the aforementioned combination partners 2.

### EXAMPLE NO 1

### Combination 1 with 2a

| | |
|---|---|
| Constituents | mg/tablet |

Core

| | |
|---|---|
| Flibanserin (free base) | 50.000 |
| Amphetamine | 70.225 |
| Anhydrous dibasic calcium phosphate | 100.000 |
| Microcrystalline cellulose | 203.090 |
| HPMC (Methocel E5) | 6.615 |
| Croscarmellose sodium | 8.820 |
| Magnesium stearate | 2.250 |

Coating

| | |
|---|---|
| HPMC (Methocel E5) | 4.320 |
| Polyethylene Glycol 6000 | 1.260 |
| Titanium dioxide | 1.800 |
| Talc | 1.542 |
| Iron oxide red | 0.078 |
| Total Film coated tablet | 450.000 |

### EXAMPLE NO 2

### Combination 1 with 2b

| | |
|---|---|
| Constituents | mg/tablet |

Core

| | |
|---|---|
| Flibanserin (free base) | 50.000 |
| Atomotoxetine | 10.000 |
| Lactose monohydrate | 133.750 |
| Microcrystalline cellulose | 40.000 |
| Hydroxypropylcellulose | 2.500 |
| Corn starch | 12.500 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide yellow | 0.043 |
| Total Film coated tablet | 255.000 |

### EXAMPLE NO 3

### Combination 1 with 2c

| | |
|---|---|
| Constituents | mg/tablet |

Core

| | |
|---|---|
| Flibanserin (free base) | 50.000 |
| Pramipexole dihydrochloride monohydrate | 1.000 |
| Lactose monohydrate | 143.490 |
| Microcrystalline cellulose | 47.810 |
| HPMC (e.g. Pharmacoat 606) | 2.500 |
| Carboxymethylcellulose sodium | 5.000 |
| Mannitol | 60.000 |
| Corn starch | 36.500 |
| Povidone | 1.000 |
| Colloidal silicon dioxide | 1.000 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated bilayer tablet | 357.000 |

### EXAMPLE NO 4

### Combination of 1 with 2d

| | |
|---|---|
| Constituents | mg/tablet |

Final Mixture

| | |
|---|---|
| Flibanserin (free base) | 50.000 |
| Ziprasidone hydrochloride monohydrate | 40.000 |
| Lactose monohydrate | 200.000 |
| Pregelatinized starch | 108.000 |
| Magnesium stearate | 2.000 |

Capsule

| | |
|---|---|
| Final Mixture | 400.000 |
| Capsule (size 1) | 82.000 |
| Total weight of Capsule | 482.000 |

The following examples show preferred pharmaceutical compositions of flibanserin, if the combinations according to the invention are administered in separate dosage units.

### EXAMPLE NO 5

### Composition

| | |
|---|---|
| Constituents | mg/tablet |

Core

| | |
|---|---|
| Flibanserin (free base) | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

### EXAMPLE NO 6

### Composition

| | |
|---|---|
| Constituents | mg/tablet |

Core

| | |
|---|---|
| Flibanserin (free base) | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

### EXAMPLE NO 7

### Composition

| | |
|---|---|
| Constituents | mg/tablet |

Core

| | |
|---|---|
| Flibanserin (free base) | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

### EXAMPLE NO 8

### Composition

| | |
|---|---|
| Constituents | mg/tablet |

Core

| | |
|---|---|
| Flibanserin (free base) | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

### EXAMPLE NO 9

### Composition

| | |
|---|---|
| Constituents | mg/tablet |

Core

| | |
|---|---|
| Flibanserin (free base) | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

### EXAMPLE NO 10

### Composition

| | |
|---|---|
| Constituents | mg/tablet |

Core

| | |
|---|---|
| Flibanserin (free base) | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

1-21. (canceled)

22) A pharmaceutical composition comprising a therapeutically effective amount of flibanserin 1, as one active ingredient, optionally in form the free base or a pharmacologically acceptable acid addition salt thereof, and a therapeutically effective amount of a second active ingredient 2 suitable for the treatment or prevention of Attention Deficit Hyperactivity Disorder (ADHD), optionally in combination with a pharmaceutically acceptable excipient.

23) A pharmaceutical composition according to claim 22, wherein the second active ingredient 2 is selected from the group consisting of adrenoceptor agonists, noradrenaline reuptake inhibitors, dopamine agonists and dopamine antagonist.

24) A pharmaceutical composition according to claim 22, wherein the second active ingredient 2 is an adrenoceptor agonist 2a.

25) A pharmaceutical composition according to claim 24, wherein the adrenoceptor agonist 2a is selected from the group consisting of Lisdexamphetamine dimesylate, Amphetamine, Dexamphetamine, NRP-104, SPD-465 (guanfacine), Modafinil and pharmaceutically acceptable acid addition salts thereof.

26) A pharmaceutical composition according to claim 22, wherein the second active ingredient 2 is a noradrenaline reuptake inhibitor 2b.

27) A pharmaceutical composition according to claim 26, wherein the noradrenaline reuptake inhibitor 2b, is selected from the group consisting of Atomoxetine, DOV-102677, DOV-216303, DOV-21947, NS 2359, NS 2330, NS 2360, NS2390 and pharmaceutically acceptable acid addition salts thereof.

28) A pharmaceutical composition according to claim 22, wherein the second active ingredient 2 is a dopamine agonist 2c.

29) A pharmaceutical composition according to claim 28, wherein the dopamine agonist 2c is selected from the group consisting of Methylphenidate, Dexmethylphenidate, SPD-485, SPD-465, SPD-483, Hydroxybupropion, Pramipexol, ABT-724, CP-226269, Dextroampthemine Bromocriptin, Cabergolin, Alpha-dihydroergocryptin, Lisuride, Pergolide, Roxindol, Ropinirol, Sopirinol, Talipexol, Bupropion, Bifemelane (SON-216), Terguride and pharmaceutically acceptable acid addition salts thereof.

30) A pharmaceutical composition according to claim 22, wherein the second active ingredient 2 is a dopamine antagonist 2d.

31) A pharmaceutical composition according to claim 30, wherein the dopamine antagonist 2d is selected from the group consisting of Olanzapine, Ziprasidone, MDL-814608A, NRA-0562, S-18126, SPI-376, YM-50001, 1192U90, ALX-D4, Balaperidone, CI-1030, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, Mianserin, Buspirone, PD-089232, PD-108306, PD-165167, PD-167063, PD-168306, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E, YM-43611 and pharmaceutically acceptable acid addition salts thereof.

32) The pharmaceutical composition according to claim 22, wherein the active ingredients 1 and 2 are together in one dosage form.

33) The pharmaceutical composition according to claim 22, wherein the active ingredients 1 and 2 are separate, each in one dosage form.

34) A method for the treatment of Attention Deficit Hyperactivity Disorder (ADHD) comprising the administration of a therapeutically effective amount of flibanserin 1, as one active ingredient, optionally in form the free base or a pharmacologically acceptable acid addition salt thereof, and a therapeutically effective amount of a second active ingredient 2, which is suitable for the treatment or prevention of Attention Deficit Hyperactivity Disorder (ADHD), optionally in combination with a pharmaceutically acceptable excipient.

35) A method according to claims 34, wherein the Attention Deficit Hyperactivity Disorder (ADHD) is selected from the group consisting of ADHD predominantly impulsivity type, ADHD predominantly inattentive type, ADHD predominantly hyperactive-impulsive type and ADHD not otherwise specified.

36) A method according to claim 34, wherein the second active ingredient 2 is selected from the group consisting of adrenoceptor agonists, noradrenaline reuptake inhibitors, dopamine agonists and dopamine antagonists.

37) A method according to claims 34, wherein the second active ingredient 2 is an adrenoceptor agonist 2a selected from the group consisting of Lisdexamphetamine dimesylate, Amphetamine, Dexamphetamine, NRP-104, SPD-465 (guanfacine), Modafinil and pharmaceutically acceptable acid addition salts thereof.

38) A method according to claim 34, wherein the second active ingredient 2 is a noradrenaline reuptake inhibitor 2b selected from the group consisting of Atomoxetine, DOV-102677, DOV-216303, DOV-21947, NS 2359, NS 2330, NS 2360, NS2390 and pharmaceutically acceptable acid addition salts thereof.

39) A method according to claim 34, wherein the second active ingredient 2 is a dopamine agonist 2c selected from the group consisting of Methylphenidate, Dexmethylphenidate, SPD-485, SPD-465, SPD-483, Hydroxybupropion, Pramipexol, ABT-724, CP-226269, Dextroampthemine Bromocriptin, Cabergolin, Alpha-dihydroergocryptin, Lisuride, Pergolide, Roxindol, Ropinirol, Sopirinol, Talipexol, Bupropion, Bifemelane (SON-216), Terguride and pharmaceutically acceptable acid addition salts thereof.

40) A method according to claim 34, wherein the second active ingredient 2 is a dopamine antagonist 2d selected from the group consisting of Olanzapine, Ziprasidone, MDL-814608A, NRA-0562, S-18126, SPI-376, YM-50001, 1192U90, ALX-D4, Balaperidone, CI-1030, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, Mianserin, Buspirone, PD-089232, PD-108306, PD-165167, PD-167063, PD-168306, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E, YM-43611 and pharmaceutically acceptable acid addition salts thereof.
